(19) United States
(12) Patent Application Publication
(10) Pub. No.: US 2014/0178468 A1
(43) Pub. Date: Jun. 26, 2014

(54) MULTIPARTICULATE EXTENDED-RELEASE COMPOSITION OF MESALAMINE

(71) Applicant: RANBAXY LABORATORIES LIMITED, New Delhi (IN)

(72) Inventors: Rajesh S. SHEAR, Gurgaon (IN); Abhishank SINGH, New Delhi (IN); Girish MITTAL, Hisar (IN); Sumit MADAN, New Delhi (IN); Vinod Kumar ARORA, Gurgaon (IN)

(73) Assignee: RANBAXY LABORATORIES LIMITED, New Delhi (IN)

(21) Appl. No.: 14/138,610

(22) Filed: Dec. 23, 2013

(30) Foreign Application Priority Data
Dec. 24, 2012 (IN) 4045/DEL/2012

(51) Int. Cl. A61K 9/50; A61K 9/48; A61K 31/196

(52) U.S. Cl. CPC A61K 9/5078; A61K 31/196; A61K 9/4833
USPC 424/461; 514/567; 264/134; 53/431

(57) ABSTRACT

The present invention relates to a multiparticulate extended-release pharmaceutical composition of mesalamine comprising a) an inert core, b) an active ingredient layer and one or more pharmaceutically acceptable excipients, c) an inner coating layer comprising a water-insoluble cellulose derivative, and d) an outer coating layer comprising an enteric polymer.

### FIELD OF THE INVENTION

The present invention relates to a multiparticulate extended-release pharmaceutical composition of mesalamine, and a process for the preparation thereof.

### BACKGROUND OF THE INVENTION

Mesalamine, 5-aminosalicylic acid, is an anti-inflammatory agent used for the treatment of mild to moderate active ulcerative colitis.

Mesalamine is commercially available in various dosage forms such as Asacol® (mesalamine 400 mg delayed-release tablets), Asacol® HD (mesalamine 800 mg delayed-release tablets), Apriso® (Mesalamine 375 mg extended-release capsules), Pentasa® (mesalamine 250 mg and 500 mg controlled-release capsules) and Lialda® (mesalamine 1.2 g delayed-release tablets) for oral administration. It is also commercially available for rectal administration as an aqueous suspension or as suppositories.

U.S. Pat. No. 6,551,620 discloses an oral pharmaceutical pellet formulation having controlled release profile, comprising a core and an enteric coating, wherein the active ingredient 5-amino-salicylic acid is present in the core in a non-gel forming polymer matrix, the matrix-forming polymer making up at least 1% by weight of the core.

U.S. Publication No. 2007/0071820 discloses a delayed-release pharmaceutical formulation comprising a drug and a disruption agent and further comprising a regulatory membrane coating on the core formed from a mixture of a water-soluble gel-forming polymer and a water-insoluble film-forming polymer.

European Patent No. EP 0 629 398 discloses a controlled-release pharmaceutical preparation comprising a core containing a medicinal compound and an enteric coating over the core, wherein the core contains a medicinal compound and an organic acid, and is film coated by an aqueous coating of water-insoluble and slightly water permeable acrylic polymer having a trimethylammonioethyl group.

### SUMMARY OF THE INVENTION

The present invention provides an alternative multiparticulate extended-release pharmaceutical composition of mesalamine, which has a dissolution profile comparable to Apriso®.

### DETAILED DESCRIPTION OF THE INVENTION

An aspect of the present invention provides a multiparticulate extended-release pharmaceutical composition of mesalamine, comprising a) an inert core, b) an active ingredient layer comprising mesalamine and other pharmaceutically acceptable excipients, c) an inner coating layer comprising a water-insoluble cellulose derivative, and d) an outer coating layer comprising an enteric polymer.

The term “mesalamine”, as used herein, is intended to include isomers, polymorphs, solvates, and hydrates of mesalamine.

Mesalamine may exist in any polymorphic form, such as crystalline or amorphous.

The multiparticulate composition is contained in a dosage form, e.g., hard gelatin capsule or a sachet, or is formed into tablets by compression. Multiparticulates can be designed to provide extended-release, delayed-release, pulsatile, or bi-phasic release, or even site-specific release of the drugs.

The term “pharmaceutically acceptable excipients”, as used herein, includes diluents, disintegrants, lubricants, glidants, anti-oxidants, plasticizers, and opacifiers.

The term “inert core”, as used herein, includes sugar spheres (non-pareil seeds) or microcrystalline cellulose beads (Celphere®). The size of the inert core ranges from about 500 μm to about 850 μm.

The drug coated core is further coated with two coating layers, i.e., an inner coat and an outer coat. The inner coating comprises a water-insoluble cellulose derivative. Examples of water-insoluble cellulose derivatives include ethyl cellulose.

Ethyl cellulose is a hydrophobic film coating agent used in pharmaceutical preparations to modify the release of drugs. Commercially available grades of ethyl cellulose like Ethocel® standard premium with different viscosities can be used. Ethyl cellulose films may be modified to alter their solubility by the addition of hypromellose or a plasticizer. The concentration of ethyl cellulose may vary from 2% w/w to 5% w/w by weight of the total composition.

Hypromellose, or HMPC, is available in various grades, for example, K4M, K15M, and K35M. When present in an ethyl cellulose layer, HPMC modifies the characteristics of the ethyl cellulose layer.

The target weight gain after applying the inner coating may vary between 5% w/w to 10% w/w by weight of the uncoated composition.

The outer coating layer comprises an enteric polymer that prevents the release of the drug in acidic conditions, and releases the drug in an alkaline environment. Examples of enteric polymers include cellulose acetate phthalate, hydroxypropylmethylcellulose acetate phthalate, polyvinyl acetate phthalate, hydroxypropyl phthalate, hydroxypropylmethylcellulose phthalate (HPMC phthalate), hydroxypropylmethylcellulose acetate succinate, methacrylic acid/methyl methacrylate copolymers, e.g., Type A (Eudragit® L 100, Eudragit® L 12.5) and Type B (Eudragit® S 100, Eudragit® S 12.5), or mixtures Thereof.

According to one embodiment of this aspect, there is provided a multiparticulate extended-release pharmaceutical composition of mesalamine, comprising a) an inert core, b) an active ingredient layer comprising mesalamine and one or more pharmaceutically acceptable excipients, c) an inner coating layer comprising ethyl cellulose, and d) an outer coating layer comprising an enteric polymer.

According to one embodiment of this aspect, the multiparticulate extended-release pharmaceutical composition further comprises a multiparticulate immediate-release composition, wherein the multiparticulate immediate-release composition comprises a) an inert core, b) an active ingredient layer and one or more pharmaceutically acceptable excipients, and c) an outer coating layer comprising an enteric polymer. The ratio of multiparticulate extended-release composition to multiparticulate immediate-release composition is from 60:40 to 90:10.

According to another embodiment of this aspect, there is provided a process for the preparation of a multiparticulate extended-release pharmaceutical composition of mesalamine, comprising the steps of:
a) dissolving/dispersing mesalamine and one or more pharmaceutically acceptable excipients in a solvent;
b) spraying the dispersion/solution obtained from step a) onto an inert core;
c) dissolving/dispersing a water-insoluble cellulose derivative and one or more pharmaceutically acceptable excipients in a solvent;
d) spraying the dispersion/solution obtained from step c) onto the mesalamine core of step b);
e) preparing a dispersion/solution of an enteric polymer along with one or more pharmaceutically acceptable excipients;
f) spraying the dispersion of step e) onto the beads of step d); and
g) lubricating the beads obtained from step f) and filling into suitable sized capsules or compressing into tablets.

Suitable diluents are selected from the group comprising calcium carbonate, calcium phosphate-dibasic, calcium phosphate-tribasic, calcium sulfate, microcrystalline cellulose, silicified microcrystalline cellulose, cellulose powdered, magnesium oxide, dextrates, dextrins, dextrose, fructose, kaolin, lactitol, lactose, mannitol, sorbitol, starch, pregelatinized starch, sucrose, sugar compressible, sugar confectioners, or mixtures thereof.

Suitable disintegrants are selected from the group comprising sodium starch glycolate, pregelatinized starch, powdered cellulose, crospovidone, croscarmellose sodium, colloidal silicon dioxide, microcrystalline cellulose, sodium carboxymethylcellulose, dried corn starch, or mixtures thereof.

Suitable lubricants and glidants are selected from the group comprising stearic acid, magnesium stearate, calcium stearate, zinc stearate, sodium stearyl fumarate, glyceryl monostearate, glyceryl palmitostearate, hydrogenated vegetable oil, mineral oil, silicon dioxide, sodium lauryl sulfate, talc, sucrose esters of fatty acid, microcrystalline wax, yellow beeswax, white beeswax, or mixtures thereof.

Suitable anti-oxidants are selected from the group comprising butylated hydroxyanisole, propyl gallate, butylated hydroxytoluene (BHT), ascorbic acid, ascorbyl palmitate, alpha-tocopherol, bioflavonoids, or mixtures thereof.

The target weight gain after enteric coating ranges between 10% and 20% by weight of the uncoated composition.

The coating composition may further comprise pharmaceutically acceptable excipients such as plasticizers and opacifiers. Examples of plasticizers include acetylated triacetin, triethyl citrate, tributyl citrate, glycerol tributyrate, diacetylated monoglyceride, polyethylene glycols, propylene glycol, sesame oil, acetyl tributyl citrate, acetyl triethyl citrate, diethyl oxalate, diethyl phthalate, diethyl maleate, diethyl fumarate, dibutyl succinate, diethyl malonate, dioctyl phthalate, dibutyl sebacate, or mixtures thereof.

Suitable opacifiers are selected from the group comprising titanium dioxide, talc, calcium carbonate, behenic acid, cetyl alcohol, or mixtures thereof.

Suitable solvents that can be used for dispersing/dissolving the coating polymer or mesalamine are selected from the group consisting of water, ethanol, methanol, ammonium hydroxide, isopropyl alcohol, or mixtures thereof.

The following examples are representative of the invention, but are not to be construed as limiting the scope of the claims.

### EXAMPLES

### Example 1

Batch A

| | |
|---|---|
| Ingredients | mg/cap |

Drug Layering

| | |
|---|---|
| Non-pareil seeds | 220.00 |
| Mesalamine | 375.00 |
| Hydroxypropylmethylcellulose E5 | 20.00 |
| Talc | 5.00 |
| Colloidal silicon dioxide | 3.00 |
| Purified water | q.s. |
| Isopropyl alcohol | q.s. |

ER Coat

| | |
|---|---|
| Ethyl cellulose | 24.05 |
| Diacetylated monoglycerides | 1.06 |
| Hydroxypropylmethylcellulose E5 | 21.50 |
| Purified water | q.s. |

DR Coat

| | |
|---|---|
| Eudragit ® L 100 | 30.00 |
| Triethyl citrate | 15.00 |
| Ammonium hydroxide | q.s. |
| Talc | 30.00 |
| Water | q.s. |
| Total | 744.61 |

Manufacturing Process:

Drug Layering
1) Non-pareil seeds were charged in a Wurster fluidized bed dryer.
2) Hydroxypropylmethylcellulose E5 was dispersed in isopropyl alcohol to form a dispersion.
3) Water was added to the dispersion of step 2).
4) Mesalamine, talc, and colloidal silicon dioxide were dispersed in the dispersion of step 3).
5) The dispersion obtained from step 4) was sprayed onto the non-pareil seeds of step 1).

ER Coat
6) Hydroxypropylmethylcellulose E5 was dissolved in water.
7) Ethyl cellulose and diacetylated monoglycerides were added to the solution of step 6).
8) The solution obtained from step 7) was sprayed onto the beads of the mesalamine core to obtain ER coated beads.

DR Coat
9) Eudragit® L 100 was dispersed in purified water.
10) The dispersion of step 9) was neutralized with ammonium hydroxide.
11) Triethyl citrate and talc were added to the dispersion of step 10) and stirred.

12) The polymeric dispersion from step 11) was sprayed onto the ER coated beads of step 8).

Batch B

| | |
|---|---|
| Ingredients | mg/cap |

Drug Layering

| | |
|---|---|
| Non-pareil seeds | 220.00 |
| Mesalamine | 375.00 |
| Hydroxypropylmethylcellulose E5 | 20.00 |
| Talc | 5.00 |
| Colloidal silicon dioxide | 3.00 |
| Purified water | q.s. |
| Isopropyl alcohol | q.s. |

DR Coat

| | |
|---|---|
| Eudragit ® L 100 | 30.00 |
| Triethyl citrate | 15.00 |
| Ammonium hydroxide | q.s. |
| Talc | 30.00 |
| Water | q.s. |
| Total | 698.00 |

Manufacturing Process:

Drug Layering
1) Non-pareil seeds were charged in a Glatt.
2) Hydroxypropylmethylcellulose E5 was dispersed in isopropyl alcohol to form a dispersion.
3) Water was added to the dispersion of step 2).
4) Mesalamine, talc, and colloidal silicon dioxide were dispersed into the dispersion of step 3).
5) The dispersion obtained from step 4) was sprayed onto non-pareil seeds of step 1).

DR Coat
6) Eudragit® L 100 was dispersed in purified water.
7) The dispersion of step 6) was neutralized with ammonium hydroxide.
8) Triethyl citrate and talc were added to the dispersion of step 7) and stirred.
9) The polymeric dispersion from step 8) was sprayed onto the beads of the mesalamine core of step 5).

Beads obtained from Batch A and Batch B were filled into suitable sized hard gelatin capsules in the ratio of 85:15.

Dissolution Study

Table 1 shows the comparison of the dissolution profiles of the formulations of Example 1 vis-à-vis Apriso®. The dissolution was performed for 2 hours in 750 mL of 0.1N HCl at 100 rpm using the basket method followed by 1000 mL of pH 6.8 phosphate buffer at 100 rpm using the basket method. The samples were analyzed using the HPLC method as described in USP (USP 29, page 1352). As seen from the results, there is no difference in the dissolution of the composition of Example 1 and Apriso®.

TABLE 1
Dissolution profile at pH 6.8 phosphate buffer/1000 mL/100 rpm

| Time point | Example | |
|---|---|---|
| (hrs) | 1 | Apriso ® |
| 0.5 | 30 | 26 |
| 1 | 40 | 41 |
| 2 | 51 | 60 |
| 4 | 80 | 83 |
| 7 | 89 | 96 |
| 9 | 90 | 101 |

We claim:

1. A multiparticulate pharmaceutical composition of mesalamine comprising
a) a multiparticulate extended-release pharmaceutical composition comprising a) an inert core, b) an active ingredient layer and one or more pharmaceutically acceptable excipients, c) an inner coating layer comprising a water-insoluble cellulose derivative, and d) an outer coating layer comprising an enteric polymer; and
b) a multiparticulate immediate-release pharmaceutical composition comprising a) an inert core, b) an active ingredient layer and one or more pharmaceutically acceptable excipients, and c) an outer coating layer comprising an enteric polymer.

2. The pharmaceutical composition according to claim 1, wherein the water-insoluble cellulose derivative is ethyl cellulose.

3. The pharmaceutical composition according to claim 1, wherein the enteric polymer is selected from the group consisting of cellulose acetate phthalate, hydroxypropyl methylcellulose acetate phthalate, polyvinyl acetate phthalate, hydroxypropyl phthalate, hydroxypropyl methylcellulose phthalate, hydroxypropyl methylcellulose acetate succinate, methacrylic acid/methyl methacrylate copolymers, or mixtures thereof.

4. The pharmaceutical composition according to claim 1, wherein the inert core is selected from the group consisting of non-pareil seeds or microcrystalline cellulose beads.

5. The pharmaceutical composition according to claim 1, wherein the size of inert core is in the range of from about 500 μm to about 850 μm.

6. The pharmaceutical composition according to claim 1, wherein the composition is a tablet, hard gelatin capsule, or a sachet.

7. The pharmaceutical composition according to claim 1, wherein the pharmaceutically acceptable excipients are selected from the group consisting of diluents, disintegrants, lubricants, glidants, plasticizers, opacifiers, and mixtures thereof.

8. The pharmaceutical composition according to claim 1, wherein the ratio of multiparticulate extended-release composition to multiparticulate immediate-release composition is from 60:40 to 90:10.

9. The pharmaceutical composition according to claim 9, wherein the ratio of multiparticulate extended-release composition to multiparticulate immediate-release composition is 85:15.

10. The pharmaceutical composition according to claim 1 wherein the extended-release composition is prepared by a process comprising the steps of:
a) dissolving/dispersing mesalamine and one or more pharmaceutically acceptable excipients in a solvent;
b) spraying the dispersion/solution obtained from step a) onto an inert core;
c) dissolving/dispersing a water-insoluble cellulose derivative and one or more pharmaceutically acceptable excipients in a solvent;
d) spraying the dispersion/solution obtained from step c) onto the beads of the mesalamine core of step b);
e) preparing a dispersion/solution of an enteric polymer along with one or more pharmaceutically acceptable excipients;
f) spraying the dispersion of step e) onto the beads of step d); and
g) lubricating the beads obtained from step f) and filling into suitable sized capsules or compressing into tablets.

11. The pharmaceutical composition according to claim 1 wherein the immediate-release composition is prepared by a process comprising the steps of:
a) dissolving/dispersing mesalamine and one or more pharmaceutically acceptable excipients in a solvent;
b) spraying the dispersion/solution obtained from step a) onto an inert core;
c) preparing a dispersion/solution of an enteric polymer along with one or more pharmaceutically acceptable excipients;
d) spraying the dispersion of step c) onto the beads of step b); and
e) lubricating the beads obtained from step d) and filling into suitable sized capsules or compressing into tablets.
